(19) United States
(12) Patent Application Publication
(10) Pub. No.: US 2004/0171970 A1
(43) Pub. Date: Sep. 2, 2004

(54) Hand-held device for pain relief

(76) Inventors: Kurt Schleuniger, Nennigkofen (CH); Hans-Jorg Zweifel, Unterengstringen (CH); Ronald Dehmlow, Tostedt (DE)

Correspondence Address: SUGHRUE MION, PLLC, 2100 PENNSYLVANIA AVENUE, N.W., SUITE 800, WASHINGTON, DC, 20037, US

(21) Appl. No.: 10/473,298

(22) PCT Filed: Apr. 2, 2002
(86) PCT No.: PCT/CH02/00183

(30) Foreign Application Priority Data
Mar. 29, 2001 (CH) 587/01

(51) Int. Cl. A61N 7/00

(52) U.S. Cl. 601/2; 601/3

(57) ABSTRACT

The invention relates to an ultrasonic therapeutic device, which can be operated independently of an external power source. For longer periods of operation, for example by a trainer at a sports ground, the device can be operated using an external battery stack and for stationary operation, the electric power can be drawn from the mains supply. For reasons of safety, the emitted ultrasonic intensity is reduced in relation to medical therapeutic devices. The same device can be used to heat or cool the area to be treated. The device can also be set to emit electrical, magnetic and electromagnetic fields and for electro-physiotherapy with or without a separate second electrode. The emission head (1) therefore contains an oscillating element (3), Peltier elements (25) for heating and cooling and the electrodes (28, 29, 30) for electro-physiotherapy.

Hand-held devices, in particular those with ultrasound and for electrotherapy, are often suitable for therapeutic use only by a physician or trained therapeutic personnel. Accordingly, they are relatively expensive and are equipped with numerous features which would be dangerous in the hands of a layperson when used incorrectly. Due to the complexity of their possible applications, these devices generally consist of an application part and a control part connected to it by a cable and designed as a desktop unit or even as a cabinet. The goal of the present invention is to create a hand-held device according to the preamble of Patent claim 1 for pain reduction, such that it can also be operated independently of the power line, and the electronics are accommodated in a housing like that of a cell phone or a shower head. Battery-operated hand-held devices are known for therapeutic devices with electromagnetic fields or stimulating current. However, the power requirements of ultrasonic devices have so far prevented construction of line-independent hand-held devices.

The area of use of the inventive device is in the field of wellness, fitness, cosmetics, pain reduction or antistress therapy in humans or for the treatment of animals.

One problem to be solved is reducing the emitted fields to the extent that they cannot be harmful, even when used incorrectly, while still achieving a therapeutic effect. This problem is solved by using a plurality of therapeutically effective signals individually or combining them together., e.g., ultrasound, electric field, magnetic field, electromagnetic field, heat or cold and stimulating current. The individual signal strengths may be varied and adjusted manually. All the therapeutic signals are emitted by a single multifunctional emitting head 1. The emitting head 1 may also contain a sensor which determines the body's response to the therapeutic signals emitted or a sensor and/or a detection device which ascertains whether the therapeutic device is emitting the therapeutic signals into the air or into a body being treated.

The essential features of the inventive device are described in the characterizing part of the independent Patent claim 1, and preferred embodiments are described in the dependent patent claims. Furthermore, a charging station according to Patent claim 18 is claimed for the inventive device.

Another problem to be solved is reducing the power consumption to the extent that the device can be operated independently of an external power source for several therapeutic sessions. This limits the choice of therapeutically effective signals which are possible for therapeutic use and determines an optimum conversion of available energy into therapeutic fields. For ultrasound in particular, it has not been possible in the past to operate therapeutic devices independently of the power line.

In the technical literature, signal strengths of therapeutic ultrasonic devices of 0.05 to 0.4 W/cm² are considered to be low, and 0.8 to 3 W/cm² is considered to be high. Therapeutic devices begin at low frequencies (10 kHz) with 0.1 mW/cm², with the effect of the ultrasound decreasing with an increase in frequency in proportion to 1/f^{1/2}. The greatest signal strengths used therapeutically are 10 mHz at 500 mW/cm² and 1 MHz in the range up to 2 W/cm².

The prevalent theory of the healing effect of ultrasound is based on the assumption that there is local heating of tissue with ultrasound. It follows from this that the healing effect disappears at a lower input of ultrasonic power because, for example, the ultrasonic energy absorbed is no longer sufficient to induce measurable heating of the tissue. However, our own medical tests have shown that, contrary to the prevailing learned opinion, pain relief is even achieved with a signal strength of <1 mW/cm² at 1 MHz. The minimum emittable ultrasonic power of 0.05 mW/cm² also brings pain relief even without the use of a gel. Because unwanted painful side effects decrease at lower therapeutic signal strengths, the overall efficacy of the device is actually improved in the range of low signal intensities. This surprising nonlinear effect makes it possible to operate ultrasonic hand-held devices even without a power line connection.

The batteries are charged in a charging station, e.g., by inductive transfer of energy. However, this same charging station may also be used to control the hand-held device and as an interface to a PC.

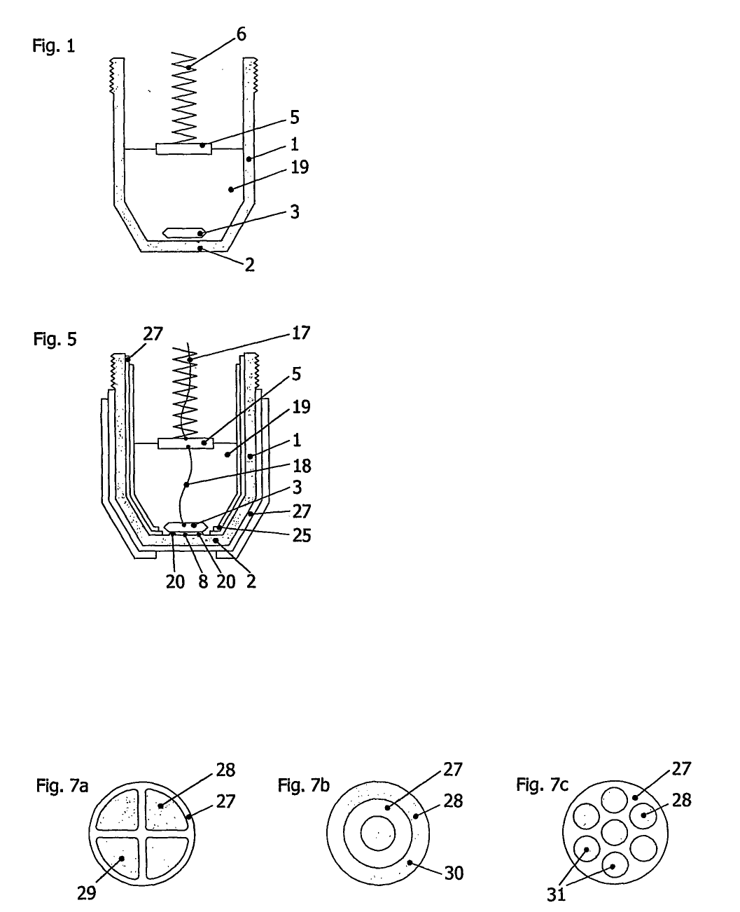

FIG. 1 shows a schematic view of a hand-held device,

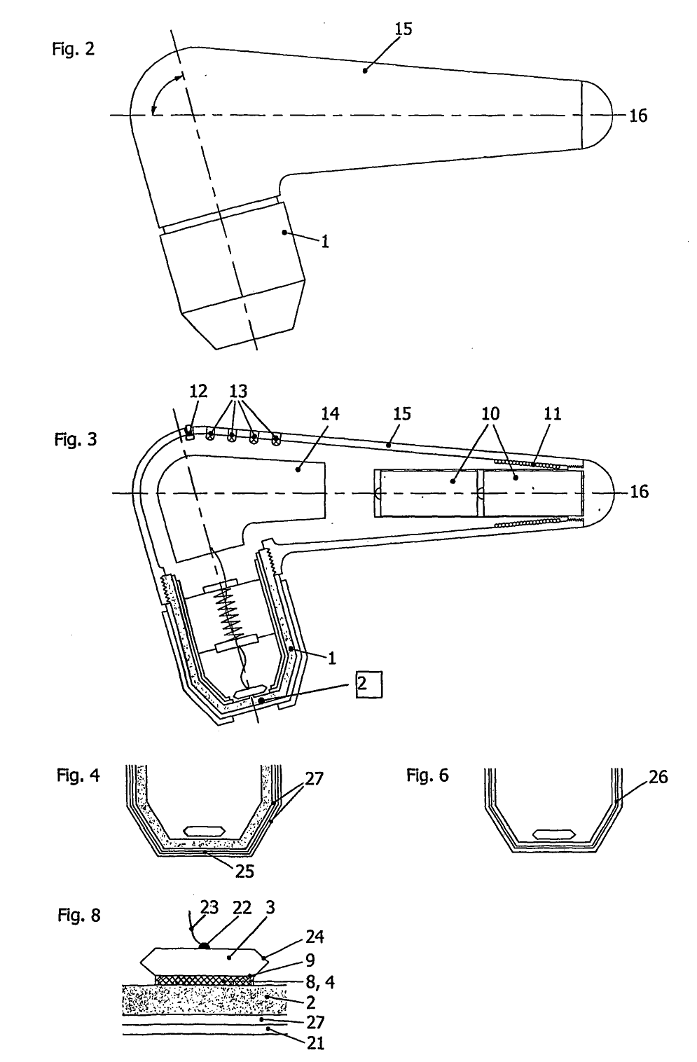

FIG. 2 shows an overall view of the hand-held device according to one exemplary embodiment of the invention,

FIG. 3 shows a section through the hand-held device according to FIG. 2,

FIG. 4 shows a detailed sectional view through the emitting head 1 with an external Peltier element 25,

FIG. 5 shows a section through the emitting head 1 with an internal Peltier element 25,

FIG. 6 shows a section through the emitting head 1 with liquid cooling,

FIGS. 7a) through c) show examples of possible electrode configurations,

FIG. 8 shows a detailed view of the emitting head 1,

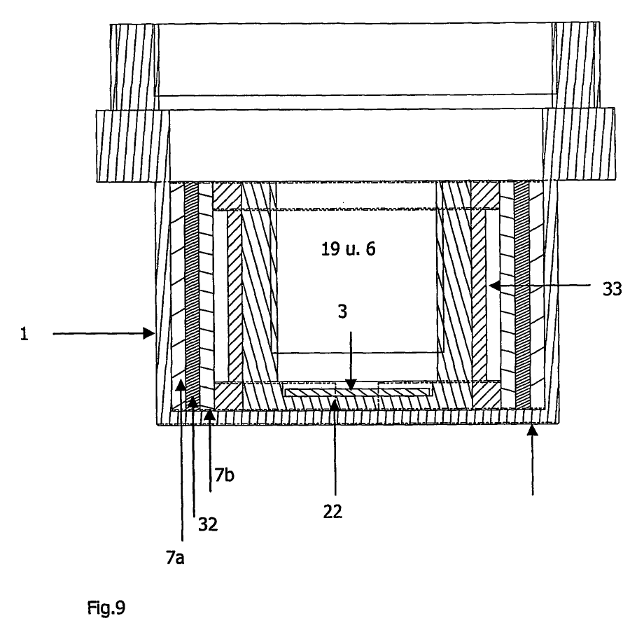

FIG. 9 shows the emitting head 1 with an integrated sensor.

FIG. 1 shows a schematic view of a replaceable emitting head 1 of a hand-held device for emission of ultrasonic therapeutic signals. An oscillating element 3 sends ultrasonic signals through a membrane 2 of an emitting head. In principle, a contact spring 6 which triggers the oscillating element via a contact plate 5 and a lower connecting wire (not shown) is sufficient for the electric triggering of the oscillating element. The electric circuit is closed across the electrically conducting wall of the emitting head. The emitting head may be screwed onto a handle part.

The overall schematic view according to FIG. 2 shows that the axis of the emitting head may form an angle other than 90° to the axis 16 of the handle 15 of the handle part. Depending on the preferred application, the angle may be less than or greater than 90°.

According to FIG. 3, there is room for the batteries 10 in the handle part of the hand-held device. If needed, however, the device may also be connected to a larger external battery pack, which may be worn on a belt or shoulder strap. For stationary use, external power packs are also provided. In the case of an external power pack, the power may be transferred inductively, so that no current-carrying parts may constitute a danger for the user, even for use in a bath or in water. The induction coils 11 are provided in the handle end of the device. This makes it possible to eliminate any sealing problems.

If a nonreplaceable emitting head is used instead of the replaceable emitting head 1, the contact spring 6 and the contact plate 5 may be omitted. Furthermore, the handle part need not be straight and conical, as shown in the schematic diagrams in FIGS. 2 and 3, but may also have a curved shape. It need only provide enough space for a battery 10, the electronic control components, etc., and it must fit in the user's hand, which is of course also possible in the case of a hand-held device in the shape of a computer mouse, for example.

The electronic control parts 14 are accommodated in the handle part of the treatment device. These include in particular the on/off switches 12 for ultrasound, stimulating current and heat or cold, electric, magnetic and/or electromagnetic fields. The electronic control unit 14 may be designed as an integrated circuit or as a microprocessor. A memory for recording treatment parameters makes it possible for a sports trainer, for example, to store the treatment parameters for different volunteers and analyze them systematically. In the simplest embodiment, the therapeutic signals may be simply switched on or off. In the comfort version, all the therapeutic signals may be used in different energy stages. In one embodiment, multiple LEDs 13 are incorporated into the handle 15 as signal displays. One LED 13 displays the type of energy emitted. In addition, the duration of the treatment may be preset. However, the integrated timer runs only when the integrated sensors detect contact with the body to be treated. When this contact is interrupted, the built-in LED 13 of the timer shuts down the timer and the LED of the blocked signal flashes. An acoustic warning signal is also emitted whenever there is a lengthy interruption, e.g., when the ultrasonic coupling is inadequate, so the ultrasonic energy can be emitted only partially into the body.

In an alternative embodiment, all the control elements are accommodated in a charging station. The hand-held device has at most one on/off switch 12. The different therapeutic signal combinations are selected on the charging station, which triggers the microprocessor in the hand-held device, e.g., optically or electrically. In charging operation, the batteries 10 accommodated in the hand-held device are charged by the charging station by inductive coupling.

FIGS. 4, 5 and 6 show details of emitting heads 1 having built-in heating or cooling with Peltier elements. Cold can be produced in the vicinity of the membrane or it may be supplied to the membrane via cooling passages 26. Hand-held devices without cooling may of course also have ohmic or semiconductor heating elements.

FIGS. 7a, 7b, 7c show examples of different forms of electrode arrangements for the stimulating current therapy and therapy with electric fields. The depth of penetration of the therapeutic signals into the body depends on the electrode configuration (sectors 29, circular rings 30, ring electrodes 31 with an external second electrode, etc.) an also depends on the triggering, i.e., the polarities selected for the individual electrodes.

FIG. 8 shows a detailed schematic view of one possible assembly of a piezocrystal as an oscillating element 3. The thickness of the material in particular has not been drawn to scale.

Furthermore, FIG. 9 shows a schematic cross section through an emitting head 3 having an integrated sensor.

This head differs from the other heads according to FIGS. 1 through 6 in which the electrodes 7a and 7b for treatment with electric fields are located inside the emitting head. The same electrodes may also be used as sensor electrodes. This emitting head is also wherein a coil is provided for generating the magnetic field, with the configuration being such that the fewest possible discrete components must be assembled so that the emitting head may fulfill all the desired functions.

A piezoceramic is used to advantage as the active piezoelectric oscillating element 3. Piezoceramic has the advantage over films and foils that it has better sympathetic oscillations, thereby facilitating optimization of the entire emitting head 1. The fundamental frequency of the ceramic is typically between 0.8 MHz and 4 MHz. However, oscillating elements 3 may also be used with frequencies between 0.5 MHz and 10 MHz. The known devices having higher frequencies up to 100 MHz are mostly used not for therapeutic purposes but instead for diagnostic purposes.

The oscillating element 3 is mounted on the membrane 2 of the emitting head 1 by using a conductive adhesive. The adhesive can fulfill four functions:

1. It secures the oscillating element 3 on the membrane 2 of the emitting head 1.

2. It permits electric contact with the lower electrode 9 of the oscillating element 3.

3. As a casting compound 4, it forms the dielectric 8 for acoustic impedance matching of the oscillating element 3 to the membrane 2.

4. As a damping medium 19, it influences the natural frequencies of the oscillating element 3 and the ultrasonic emission parallel to the membrane 2 and vertically away from the membrane 2.

In a special type of embodiment, one or more elevations 20 on the membrane 2 ensure that the thickness of the casting compound 4 corresponds to the target value at all points. Due to its chemical composition, adjusted thickness and admixtures, the casting compound 4 forms a λ/4 layer which ensures impedance matching of the oscillating element 3 to the membrane 2 of the emitting head 1 and to the skin of the user. The admixture used may be, for example, a powder of metal dust, ceramic or glass. The conductive adhesive 22, the casting compound 4 and the damping medium 19 may be made of the same material, which fulfills different functions depending on its location in the emitting head 1.

A relatively thick sheet, e.g., 1 mm thick, may be used as membrane 2 because there must not be any macroscopically detectable deformation of the membrane 2 with the ultrasonic vibration. It is important only that the ultrasonic waves must be able to penetrate through the membrane 2 without significant damping. The exterior side of the membrane 2 may also be designed as a spherical cup, so that contact with the surface of the body to be treated is also ensured in a part of the membrane 2.

The side of the membrane 2 facing the body of the user may also be covered with a X/4 layer 21, so that transfer of ultrasonic energy from the membrane 2 to a body is optimized.

The upper electrode of the oscillating element 3 is contacted with a conductive adhesive 22 or a spring 6.

This prevents formation of a dead point in soldering, i.e., a point where the piezo material has lost its piezoelectric property because of overheating.

The rear side of the oscillating element 3 is embedded in a casting compound 4 made of plastic, epoxy or Araldit. The ceramic is therefore protected from mechanical shock, and the ultrasonic waves emitted to the rear are attenuated.

In a preferred embodiment, the sides of a circular piezoceramic are not straight but instead are inclined with a double facet (see FIG. 8). In the case of circular oscillating elements 3, the largest circumference of the circle is at half-height. The ceramic is also mounted laterally in the casting compound 4 to dampen unwanted longitudinal vibrations.

These measures achieve the result that the oscillating element 3 oscillates at a single frequency, and maximum ultrasonic energy can be emitted with minimal energy loss with vibrating membrane 2.

As an alternative, the oscillating element 3 may be constructed and installed in such a way that it includes the broadest possible oscillation spectrum without any sharp resonance, so that the signal frequencies emitted can be adjusted, e.g., by the microcontroller, without any change in the mechanical design.

Some of the pain that can be treated with this hand-held device is relieved by the influence of heat, and some is relieved by the influence of cold. These facts can be taken into account by using a Peltier element 25 as the heating and cooling element. To dissipate the heat generated by the electronics and the ultrasonic element in the cooling mode, a cooling medium is pumped through cooling passages 26 using a micropump.

For example, the surface of the emitting head 1 or the handle 15 may be used as the cooling surface. The maximum heating temperature is 40° C. and the lowest temperature of the membrane 2 is 5° C. If cooling is omitted, a resistor or a semiconductor may be used as the heating element.

The entire hand-held device should be designed to be waterproof. This achieves the result that even massaging in the bath can be allowed. The permanently installed battery 10 is charged inductively by a charging station. Likewise, only inductive coupling via the induction coils 11 is achieved in operation on a power line. In operation with an external battery pack, the direct voltage must first be transformed before it can be transmitted to the hand-held device.

The ultrasonic power emitted is advantageously modulated. The modulation may be saw-tooth, square-wave or sinusoidal. A single pulse packet may include only a single ultrasonic pulse or a plurality of pulses. The burst may last for 300 nanoseconds (at 3 MHz ultrasound) up to 1 second. A packet is typically followed by a pause of the same duration, i.e., the duty cycle usually amounts to 50% or more. This permits a longer use time in operation independently of the power line while also preventing the risk of tissue damage in the event of improper use. However, a range from 10% to 75% or continuous adjustability from 0% to 100% would also be conceivable. For the same reason, the maximum emitted ultrasonic intensity may be limited to 80 mW/cm². The minimum emitted ultrasonic intensity is 0.05 mW/cm².

If the membrane 2 is lifted up from the body during the treatment, then the ultrasonic waves generated are mostly reflected at the interface between the membrane 2 and air. This can be detected by the electronic controller 14. The LED 13, which indicates emission of ultrasonic waves, begins to flash immediately and the timer for the duration of the treatment is stopped. After 10 seconds, an acoustic warning signal sounds, and after another 10 seconds the treatment is automatically stopped. This feature may be deactivated or omitted in devices designed for use without a gel.

The intensity, i.e., the voltage or the current in stimulating current therapy, can only be adjusted individually. A given voltage may be hardly perceptible to a user with dry skin but unpleasant to another user with moist skin. It is therefore conventional to specify neither the voltage nor the current to be used in stimulating current therapy. Instead, the user selects the range suitable for him or her. The electronic regulating mechanisms for stimulating current therapy ensure only that there is no voltage which would result in unpleasant, let alone harmful, amperage when stopping use or with renewed contact with the body.

The amperage emitted for the stimulating current therapy is preferably in the range of 0.1, 1 or 10 mA.

As in the case of the ultrasonic signal, poor contact leads after 3 seconds to flashing of one of the signal displays 13 and after 10 seconds the device is turned off.

Stimulating current therapy is impossible for use underwater. The stimulating current electrodes (28, 29, 30, 31) would be short-circuited in water. Again in this case, one of the signal displays 13 will flash first for three seconds, and then after 10 seconds the signal is automatically shut down.

As an additional measure, the electromagnetic radiation generated by excitation of the piezoelectric vibrating element 3 may be only partially shielded. This permits stimulation of the tissue to be treated with electromagnetic fields. To do so, the housing of the emitting head 1 is designed to function as a shield, for example, except for the membrane-side end. This membrane side may be made in part of a nonshielding material, so that in some areas the electromagnetic radiation can be emitted unhindered or the shielding may be less efficient so that an attenuated radiation is emitted over the entire area. The flux density of the magnetic field preferably varies on the order of 1, 10 or 100 μT. An electric field strength on the order of 0.5, 1, 2 or 4 V/m is proposed. For the emission of the fields, an antenna, a coil 33, a capacitor plate, a film or two cylindrical electrodes 7a, 7b may be integrated into the head part, so that electric, magnetic or electromagnetic fields may be emitted individually or in combination.

Furthermore, sensors which detect the response of the treated body to the incident therapeutic signals may also be integrated into the emitting head part. As a possible simple embodiment, two cylindrical electrodes 7a, 7b are proposed, surrounding the other components of the emitting head 1. The same two electrodes 7a, 7b may also be used to generate the electric field.

In the small form, the diameter of the ultrasound-emitting membrane 2 is 5 or 10 mm, and in the large form, it is 30 mm. The small embodiment is recommended for treatment of joints, sprained fingers or toes, for example, while the large embodiment is recommended for treatment of larger flat parts of the body. In the simplest embodiment, a small ceramic oscillating element 3 is used for ultrasonic stimulation. To enable the focusability of the ultrasonic waves penetrating into the body, a plurality of ceramic rings arranged concentrically or a configuration of multiple ring electrodes 31 may be used.

The angle α between the axis of the sound-emitting head and the axis of the handle 15 may be 90° or less or more than 90°, depending on the preferred type of use of the device. The same angle is not ideal for treatment of one's own back or for the body of another person. The angle is adjustable in the medical embodiment. If the sound head is designed to be spherical, then contact with the spherical cup-shaped membrane 2 is automatically ensured in a certain angular tolerance.

A shock-absorbing pin, a spiral contact spring 6 or a simple contact wire 23 may be used for contacting the oscillating element 3. When using a contact plate 5 on the damping medium 19, the contact wire 23 is divided into two parts, e.g., an upper connecting wire 17 and a lower connecting wire 18. The oscillating element 3 may be contacted with simple mechanical contact, with a conductive adhesive 22 or by soldering. The conductive adhesive 22 then combines the advantage of reliable contact and thermal stability.

In soldering, either a low-melting solder having a low thermal stability must be used or local depolarization of the piezoelectric oscillating element 3 must be tolerated.

All the therapeutic fields emitted by the hand-held device except for the thermal signal may depend directly on the oscillation frequency of the piezoelectric element or may be stepped down by a frequency divider. Furthermore, they may be modulated at 1 Hz, 30 Hz, 50 Hz or 100 Hz or not modulated at all.

In simultaneous delivery of different therapeutic signals, their defined frequencies, intensities and signal shapes are optimally coordinated in a combination so as to achieve maximum efficacy.

The function of the charging station is to recharge the batteries 10 of the hand-held device. However, it may also assume other functions. In particular, it may be designed so that the therapeutic signal parameters can be adjusted on the charging station and transmitted optically or electromagnetically to the hand-held device. On the other hand, signals of a sensor integrated into the hand-held device may be read out by the charging station and analyzed as needed or transmitted to a PC. Then the charging station functions as a control unit or as an interface.

1 emitting head

2 membrane

3 oscillating element

4 casting compound

5 contact plate

6 contact spring

7a first electrode for the electric field, external cylindrical electrode, measurement electrode

7b second electrode for the electric field, internal cylindrical electrode, measurement electrode

8 dielectric

9 lower electrode

10 battery

11 induction coils for charging

12 on/off switch

13 signal displays, LEDs

14 electronic control unit

15 handle

16 axis of the handle

17 upper connecting wire

18 lower connecting wire

19 attenuating medium

20 elevations

21 λ/4 layer

22 conductive adhesive

23 contact wire

24 side face

25 Peltier element

26 cooling ducts

27 insulation layer

28 stimulating current electrode

29 sectors

30 concentric rings

31 circular electrodes

32 dielectric spacer

33 coil for the magnetic therapeutic signal

What is claimed is:

1. A hand-held device, in particular for therapeutic treatment with ultrasound, wherein it includes a handle (15) and an emitting head (1) for emission of ultrasonic waves, whereby a piezoelectric crystal, a piezoceramic or a piezoelectric film is mounted on the rear side of a membrane (2) as the active piezoelectric oscillating element (3) in a housing of the emitting head (1); the hand-held device can be operated independently of an external power source; and it has a timer so that the duration of the treatment can be preset.

2. The hand-held device as claimed in claim 1, wherein the membrane (2) can be heated and/or cooled so that the membrane temperature can be regulated between normal body temperature and a maximum of 40° C. and/or the membrane temperature can be regulated at any desired temperature between normal body temperature and 5° C.

3. The hand-held device as claimed in claim 2, wherein a resistor or a semiconductor is used as the heating element and/or a Peltier element (25) is used for heating and cooling, the element being applied directly to an insulation layer (27) of the membrane (2) on the inside or outside, with a fluid flowing through the emitting head (1) for transport of heat.

4. The hand-held device as claimed in any one of claims 1 through 3, wherein it is also suitable for stimulating current therapy and the membrane (2) has an electrically conducting surface so that it can function as a stimulating current electrode (28, 29, 30, 31), an electronic unit being used for regulating the current or voltage.

5. The hand-held device as claimed in claim 4, wherein a second stimulating current electrode (28, 29, 30, 31) is designed as a concentric ring (30) or as a surface around the membrane (2), or the pin electrodes arranged in a ring around the membrane (2) are used as the stimulating current electrodes (28) or one or more external stimulating current electrodes are used as the second stimulating current electrode.

6. The hand-held device as claimed in claim 4, wherein first and second stimulating current electrodes (28) are designed as partial areas of the membrane (2), and these partial areas are designed as sectors (29), concentric rings (30) or as an arrangement of circular electrodes (31).

7. The hand-held device as claimed in any one of claims 4 through 6, wherein the amperage for the stimulating current therapy may be regulated in the range from 0 to 500 mA, in particular 10 to 100 mA or 0.1 to 10 mA, and individual electric pulses of a square-wave, triangular or sinusoidal shape may be emitted individually or in bursts.

8. The hand-held device as claimed in any one of claims 1 through 7, wherein a piezoceramic is used as the active oscillating element (3), and the ultrasonic intensity emitted is in the range of 0.05 to 1000 mW/cm² in particular in the range of 0.5 to 200 mW/cm² or in the range of 10 to 80 mW/cm² and/or the active oscillating element is mounted on the membrane (2) with a conductive adhesive (22) and/or a casting compound (4) forms a λ/4 layer between the oscillating element (3) and the membrane (2) and/or the oscillating element (3) has a double-faceted side face (24); the ultrasonic intensity is emitted continuously or is modulated with a saw-tooth, square-wave or sinusoidal waveform and/or the rear side of the oscillating element (3) is embedded in an attenuating medium (19) and/or the side of the membrane (2) facing the user's body is also finished with a λ/4 layer (21).

9. The hand-held device as claimed in any one of preceding claims, wherein an external power pack or battery pack may be used as the power source; the hand-held device may also be used underwater and/or the emitting head (1) can be tilted about an axis running across the (16) of the handle; the therapeutic signal emission is reduced to a minimum if the ultrasonic coupling or field coupling to a body is stopped, the timer is stopped and/or an acoustic or optical warning signal is delivered.

10. The hand-held device as claimed in any one of preceding claims 1 through 9, wherein the membrane-side end of the housing of the emitting head (1) is partially or completely permeable for other defined therapeutic fields, in particular for electric, magnetic or electromagnetic fields, and a film, a plate, ring electrodes or cylindrical electrodes (7a, 7b) or a coil (33) or an antenna for emission of the electric or magnetic or electromagnetic field is integrated into the emitting head.

11. The hand-held device as claimed in claim 10, wherein the strength of the electric field that can be emitted is 0 V/m or 0.5 to 4 V/m, in particular 1 to 2 V/m, and the flux density of the magnetic field that can be delivered is 0 μT, 1 μT to 100 μT, in particular 1 μT to 10 μT.

12. The hand-held device as claimed in any one of claims 1 through 11, wherein the different therapeutic fields can be activated, controlled or regulated from outside of the hand-held device.

13. The hand-held device as claimed in any one of claims 1 through 12, wherein the therapeutic fields are modulated in the same or different ways, in particular at 1 Hz, 30 Hz, 50 Hz or 100 Hz.

14. The hand-held device as claimed in any one of claims 1 through 13, wherein the fundamental frequency of the electric, magnetic or electromagnetic fields emitted is obtained by frequency dividing or multiplication from the frequency of a quartz signal or it is predetermined by a microcontroller.

15. The hand-held device as claimed in any one of claims 1 through 14, wherein a sensor is integrated into the emitting head (1) so that the response of the body treated to the therapeutic signals can be ascertained.

16. The hand-held device as claimed in claim 15, wherein the sensor includes two cylindrical measurement electrodes (7a, 7b) which surround the other components of the emitting head (1), e.g., the aforementioned oscillating element (3), the aforementioned coil (33) and the aforementioned plate, and measure the impedance of the body thus treated.

17. The hand-held device as claimed in claims 10 and 16, wherein the cylindrical measurement electrodes (7a, 7b) may also function as the antenna for emission of the electric field.

18. A charging station for a hand-held device as claimed in any one of claims 1 through 17 for supplying power to the hand-held device.

19. The charging station as claimed in claim 18, wherein the power is transmitted inductively from the charging station to the hand-held device and the therapeutic signal parameters are sent on the charging station and can be transmitted optically or electromagnetically to the hand-held device.

20. The charging station as claimed in any one of claims 18 or 19, wherein it is set up for data transmission to and from the hand-held device.
